(19) United States
(12) Patent Application Publication
(10) Pub. No.: US 2009/0137919 A1
(43) Pub. Date: May 28, 2009

(54) LUNG VOLUME MONITORING METHOD AND DEVICE

(75) Inventors: Yaron Bar-Lavie, Zichron Yaakov (IL); Noam Gavriely, Haifa (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: Technion City, Haifa (IL)

(21) Appl. No.: 12/064,963

(22) PCT Filed: Sep. 3, 2006
(86) PCT No.: PCT/IL2006/001021

§ 371 Date: Aug. 25, 2008

Related U.S. Application Data
(60) Provisional application No. 60/713,355, filed on Sep. 2, 2005.

(51) Int. Cl. A61B 5/08

(52) U.S. Cl. 600/538

(57) ABSTRACT

A method for determining functional residual capacity (FRC) of a patient while ventilating the patient. The system may include a medical ventilator which provides inhalation and exhalation from the patient, a sensor which measures a fraction one or more gas components of the inhalation and a fraction of the same gas components of the exhalation. A step change of oxygen fraction is provided to the inhalation of the patient. Subsequent to the step change, The fractions of the gas components are measured in the inhalation and in the exhalation. The functional residual capacity of the lungs of the patient is measured based on the fraction of the gas components in the inhalation and in the exhalation. The step change is provided manually by a technician, or automatically by programming a programmable device to provide the step change automatically to the patient by the medical ventilator. The step change is either an increase or a decrease in of the oxygen fraction.

### FIELD OF THE INVENTION

The present invention relates to the field of medical devices. More particularly, the present invention relates to a novel method and device for measuring functional residual capacity (FRC) of the lungs.

### BACKGROUND OF THE INVENTION

Respiratory failure is a medical term used to describe inadequate gas exchange by the respiratory system. Respiratory failure can be indicated by observing a drop in blood oxygen level (hypoxemia) and/or a rise in arterial carbon dioxide (hypercapnia). Respiratory failure is caused by various illnesses related to muscle control, especially illnesses related to respiratory muscles. Respiratory failure can be caused by neurological diseases or injuries, when the brain is unable to activate the mechanical system responsible for the breathing.

Respiratory failure can also be caused by illnesses such as pneumonia, in which the alveoli (microscopic air-filled sacs of the lung responsible for absorbing oxygen from the atmosphere) become inflamed and flooded with fluid. Pneumonia results from a variety of causes, including infection with bacteria, viruses, fungi, or parasites, and may also occur from chemical or physical injury to the lungs, or indirectly due to other medical illnesses, such as lung cancer or alcohol abuse.

Other infectious illnesses involve severe secretion accumulation, necrosis or inability to oxidize the internal surface of the alveoli may be responsible for a respiratory failure.

Emergency treatment for respiratory failure generally includes mechanical ventilation which is required to assist or replace spontaneous breathing. Most ventilators receive pressurized oxygen from a compressed oxygen source such as a pressurized balloon and combine the oxygen with either atmospheric air or with a pressurized air source. The combination results in inhaled gas or inhalation (which has oxygen percentage different than the atmospheric concentration of oxygen, which is 21% oxygen. Depending on the situation, mechanical ventilation may be continued for a few minutes or even many years. There are a variety of ventilators which are used in different situations, such as:

a. Intensive Care Units (ICU) ventilators—sophisticated ventilators with a variety of ventilation modes that are able to be tuned according to the patient's needs.

b. Portable ventilators—are used in army units, helicopters, patients that need mobility.

c. Home care ventilators are used mainly for chronic patients. Home care ventilators accompany the patient for many years, and must have a good battery performance (in order to allow the ambulatory patient to go out and continue daily activities)

d. Pediatric/neonate ventilators are used especially for fast and accurate breath patterns characteristic in newborns and young children.

Integrated features within the ventilator, which allow the physician to program the different parameters of the ventilation according to the patient's needs are, the levels of O₂%, rate of delivering O₂, duration of each pulse of O₂, number of breaths per minute between each pulse of O₂, volume of each breath, maximal inspiratory and end-expiratory pressure. While a patient is on a ventilator, measurements are required in order to assess the condition of the lungs and the respiratory system. FRC (functional residual capacity) is the amount of air present in the lungs at the end of passive expiration and is a critical variable which indicates whether the lungs are able to deliver enough oxygen during gas exchange process. If only half of the lung function during the respiratory process then an acute hypoxemia (a lack of oxygen in the blood) occurs. If only a small part of the lungs function, then extreme hypoxemia is expected, therefore the sooner the physician knows about a volume reduction, the better it can be treated.

As mentioned above, functional residual capacity (FRC) provides information on the availability of lung surface for gas exchange FRC indicates the condition of the lungs and the respiratory system, or the presence of obstruction and secretions in the respiratory system.

FRC provides essential information useful to determine the beneficial effect of therapeutic modalities such as Positive End of Expiratory Pressure (PEEP), positional shifts of the patient, recruitment maneuvers, and also for examining the compatibility of the ventilator parameters to the patient's needs.

PEEP is a parameter adjusted by a ventilator which keeps a predetermined pressure inside the lungs at the end of the expiration preventing the alveoli from collapsing. The pressure at the end of the expiration is measured and carefully adjusted by the operator. It is important to control the PEEP continuously (at the end of each expiration) because high levels of PEEP may cause complications. Basically the goal is to maintain a minimal pressure sufficient to prevent the alveoli from collapsing.

Regarding positional shifts of a patient, it is sometimes beneficial to move the patient in positions that change the gravity effects on the lungs. The secretion accumulation as well as other physiological processes are gravity dependent.

In order to recruit more alveoli for the oxygenation process it is essential to clear as many of them as possible (from secretion, infection, mucus etc. . . . ) and to increase the availability of lung surface for gas exchange. A constant pressure is applied to the lungs between breaths. In recruitment maneuver, the pressure inside the lungs increases to more than five times of the regular pressure, expanding the alveoli and allowing O₂ to come in contact with the alveolar surface.

Other recruitment maneuvers measurements include using inert gases, for example, nitrogen or helium. By adding these gases we can monitor the amount of O₂ being exhaled or inhaled.

Moreover, FRC can provide information on the effect of the therapeutic modalities described above and also on the effect of a counter reaction. A counter reaction is accomplished by changing the ventilation parameters, for example, when the fraction of inhaled oxygen (FiO₂) drops a few percent, a counter reaction may be an increase of PEEP, an increase in O₂% or perhaps a change in the ventilation mode. The fraction of exhaled oxygen (F_{E}O₂) is also measured by oxygen sensors

Current methods for evaluating respiratory system function are deduced by direct or indirect processes. A Direct method known today which measures the FRC and often require placing the patient in a plethysmograph, and is not always feasible for a patient on a ventilator. The use of a plethysmograph requires submerging the patient in water, ventilating him and monitoring the residual volume. FRC is calculated using a plethysmograph, by calculating the residual volume when partially releasing the volume inside the lungs and measuring the amount of water that is displaced. However, it is very difficult to implement this method on critically ill patients, who cannot be moved, for example, a head injured patient. Other methods for measuring the FRC use inert gas washout or dilution which does not participate in O₂ penetration through the lungs. Therefore it is possible to create a mixture of typically only three gases in the lungs: Nitrogen (or Helium), O₂ and CO₂, and monitor them. However, monitoring Nitrogen and Helium requires special and expensive equipment.

Indirect methods of lung assessment include a determination of a pressure-volume curve (P-V) of the lungs and a calculation of the dynamic compliance and resistance.

A pressure volume curve is acquired by using a narrow endotracheal tube which is entered into the lungs through the trachea. A dynamic compliance calculation is derived from the P-V curve. A dynamic compliance calculation is often inaccurate due to obstructions in the endotracheal tube, inhomogeneity of lung mechanical properties or the presence of secretions in the airways.

Thus, there is a need for and it would be advantageous to have a device and method for measuring functional residual capacity (FRC) repeatedly and frequently following therapeutic modalities such as lung-recruitment maneuvers essential for accurate and successful management of critically ill ventilated patients. The present invention is a method and device which provides accurate FRC measurements without moving the patient's body, nor is it subject to obstructions or inhomogeneity of the lungs.

The term “concentration” as used herein refers to a volume fraction of one or more components of a gas mixture, typically in percent.

The term “tidal volume” as used herein is the fraction of volume which actually reaches the alveolar zone and is determined by measurements of volumetric flow.

The term “end tidal concentration” as used herein of a component is defined to be the component's concentration measured at the end of each breath. An analyzer may determine the concentration levels of the component in each breath.

The terms “inhalation” and “exhalation” are used herein to refer to the gas mixture inhaled and exhaled respectively by a patient during ventilation.

The terms “inhalation” and “inspiration” are used herein interchangeably.

The terms “exhalation” and “expiration” are used hereinafter interchangeably.

The term “component” as used herein in the context of inhalation (inhaled gas) and exhalation (exhaled gas) during ventilation refers to one or more gas components, such as oxygen, nitrogen or carbon dioxide.

The term “technician” as used herein refers to an operator of a method of the present invention including medical personnel, physicians, nurses and the like.

### BRIEF DESCRIPTION OF THE INVENTION

There is thus provided, in accordance with some preferred embodiments of the present invention, a device for measuring the functional residual capacity (FRC) of the lungs. According to the present invention there is provided a method for determining functional residual capacity (FRC) of a patient. The patient exhales into the system providing exhalation to the system and the patient inhales inhalation provided by the system. The system includes a sensor which measures a fraction of one or more gas components of the inhalation and a fraction of the same gas components of the exhalation. A step change of oxygen fraction is provided to the inhalation of the patient. Subsequent to the step change, the fractions of the gas components are measured in the inhalation and in the exhalation. The functional residual capacity of the lungs of the patient is measured based on the fraction of the gas components in the inhalation and in the exhalation.

The step change is provided manually by a technician, or automatically by programming a programmable device to provide the step change to the patient by the medical ventilator. The step change is either an increase or a decrease in the oxygen fraction. The amplitude of the step change is preferably based on the percentage of oxygen provided before the step change. The step change (percentage of oxygen in total volume of inhalation) is preferably between 25% minimal change and 79% maximal change. As noted previously a step change is an increment or a decrement in the percentage of an oxygen level. The duration of the step change is preferably between 10-15 breaths of the patient. It is an option to provide multiple step changes, wherein, the time duration between the step changes is about five seconds or between three and seven seconds. Preferably, the gas component (being measured in the inhalation and exhalation includes oxygen and/or carbon dioxide and the components are measured using volumetric flow measurements. The fractions of the gas components are measured using gas sensors, e.g. an oxygen sensor and/or a carbon dioxide sensor.

Inhaled and exhaled volumetric flow is measured by a flow transducer and/or by a hot wire anemometer. Calculating the functional residual capacity of the lungs is performed by calculating a tidal volume of a breath of the patient and/or by calculating initial and final nitrogen volume fractions; or the tidal volume is calculated derived from the volumetric flow measurements. Preferably, the initial and final nitrogen concentrations are determined by measuring an end tidal nitrogen concentration, and the functional residual capacity of the lungs is calculated using initial and final nitrogen quantities. Preferably, the functional residual capacity is calculated based on the difference between an exhaled nitrogen quantity and an inhaled nitrogen quantity and the difference between the exhaled nitrogen quantity and the inhaled nitrogen quantities includes measuring a nitrogen quantity difference between the exhalation and inhalation during a transition. Preferably, the difference between the exhaled nitrogen quantity and the inhaled nitrogen quantity is calculated based on collecting exhaled nitrogen, during a transition, from the onset of the step change until the nitrogen level is constant.

According to the present invention there is provided, a system for determining functional residual capacity (FRC), the system include a medical device which provides inhalation to the patient, a sensor which measures a fraction one or more components of the inhalation and a second fraction of the one or more components of exhalation from the patient. A step change of oxygen fraction of the inhalation is provided to the patient, and subsequent to the step change, the fraction of the components in the inhalation is measured and the second fraction in the exhalation is measured. The functional residual capacity of the lungs of the patient is calculated based on the fraction and the second fraction. Preferably, a programmable device provides the step change to a medical ventilator. The programmable device receives a signal as output of the sensor, and based on the output and the step change, calculates the functional residual capacity. The medical device is preferably integrated with a medical ventilator.

### BRIEF DESCRIPTION OF THE FIGURES

In order to better understand the present invention, and appreciate its practical application, the following Figures are provided and are referenced hereafter. It should be noted that the Figures are given as examples only and in no way limit the scope of the invention. Like components are denoted by like reference numerals.

FIG. 1 illustrates a block diagram of a method, according to embodiments of the present invention;

FIG. 2 is a graph, according to embodiments of the present invention illustrating a step stimulus in oxygen in the inhalation and the response to the step in the expiration;

FIG. 3 illustrates three monitoring readings, according to an embodiment of the present invention, of a flow reading the volume of air which flows from one point to a second in a predetermined time and concentration level readings of oxygen and carbon dioxide;

FIG. 4 illustrates a plot, according to embodiments of the present invention, of O₂, CO₂ and N₂ quantities in [cc] as a function of the patient's breaths; and

FIG. 5 illustrates a schematic diagram when using a ventilator, according to embodiments of the present invention of the FRC device.

FIG. 6 illustrates a schematic diagram when the patient is able to breath spontaneously, according to embodiments of the present invention of the FRC device

### DETAILED DESCRIPTION OF THE PREFERRED EMBODIMENTS

The present invention is of a system and method for monitoring the lung volume, by measuring the FRC of a patient. Specifically, the system and method enables a physician to determine the FRC accurately without a need to shift the patient or to cause him any stressful maneuvers.

The principles and operation of a system and method for monitoring the lung volume, by measuring the FRC of a patient, according to the present invention, may be better understood with reference to the drawings and the accompanying description.

Before explaining embodiments of the invention in detail, it is to be understood that the invention is not limited in its application to the details of design and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

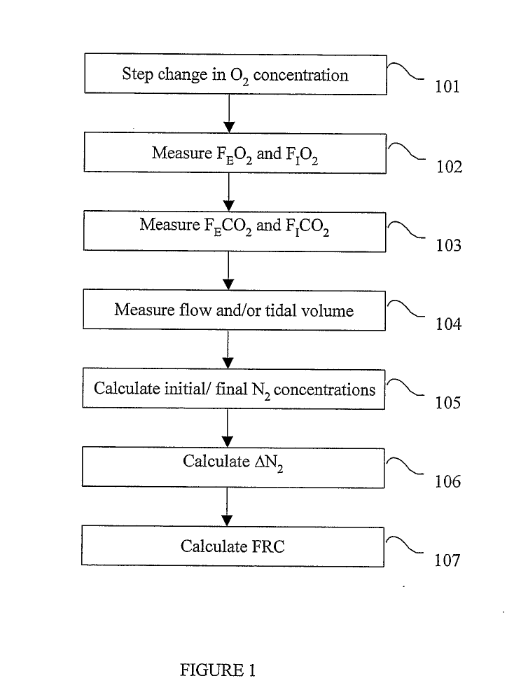

Reference is made to FIG. 1, a block diagram illustrating a procedure of measuring FRC. In step 101, a sudden step change of the concentration of the inhaled oxygen (FiO₂) is generated, which may be an increase or a decrease, in the FiO₂.

The percentage of oxygen is typically controllable in prior art ventilators by mixing different amounts of oxygen with air or other gases. There are two options for controlling the O₂% supplied to the patient. In the first option O₂% is manually changed. This would be the case when ventilators lack the internal controls or an interface to an external device using a specific preferably standard communications protocol. The second option uses an integrated feature within the ventilator, which allows the physician to program the step according to the preferred features. The amplitude of the step change is determined according to the percentage of oxygen provided before the step change. If a patient has a steady condition when provided with specific oxygen percentage, for example 60% oxygen, the step will usually be an increase in the oxygen percentage, rising to even 100% oxygen. In case the patient is on 100% oxygen the step would be a decrease in the oxygen percentage, reducing to 60% or 40%. The step occurs for at least 10-15 breaths, after the step change, the mixture of air supplied to the patient returns back to the initial mixture with the initial oxygen percentage. The sudden step changes do not affect the arterial saturation of a patient. It usually takes 1-2 minutes before detecting a change in the arterial saturation. Raising the percentage in oxygen concentration is always preferable due to medical hazards, but in case the patient is already on 100% oxygen, the step has to be a decrease in the oxygen concentration, for a brief period of time

After a step change is generated (step 101), the concentrations and quantities of the exhaled and the inhaled oxygen is measured in step 102 and the concentration of exhaled and inhaled carbon dioxide concentration levels are optionally measured in step 103 by sensors. The carbon dioxide concentration and quantity usually do not change during the step change. It is preferable that during step 103 FiO₂ is kept constant and that the tidal volume is stable for the entire duration of data acquisition.

Volumetric flow is measured in step 104. Data is provided to calculate the tidal volume for each breath. In order to improve reliability and reduce costs, a differential flow transducer is preferably used for flow acquisition. The flow transducer receives from both, inhalation and the expiration tubes and pass the air in both direction, to and from a patient. The transducer can distinguish between inhalation and expiration, each type of expiratory action (inhalation or expiration) varies by the differential pressure pattern. Therefore a tidal volume is determined for inhalation and exhalation separately. It is important to measure in both directions to verify that there aren't any leaks in the system. A simple algorithm for calculating tidal volume will be incorporated within the device's microprocessor. A hot wire anemometer (flow meter) is another option for flow measurement.

Nitrogen concentration (FN₂) is calculated (step 105) by directly measuring the end tidal nitrogen concentration with a nitrogen analyzer or determined indirectly by Equation 1. The oxygen concentration at a given time is FO₂ and the carbon dioxide concentration at a given time is FCO₂.

FN₂=0.99−(FO₂+FCO₂) Equation 1

The end tidal concentration of nitrogen is calculated at the end of each breath. Since O₂, N₂ and CO₂ are the only gases in the lungs, by measuring O₂ with a capnograph or an O₂ sensor, the nitrogen quantity can be calculated by Equation 2.

CO₂ is usually constant in all readings, CO₂ may be measured by a sensor when is not constant.

T.V.=CO₂[cc]+N₂[cc]+O₂[cc] Equation 2

Thus, nitrogen concentration levels can be determined at any time when the corresponding values of oxygen and carbon dioxide concentration levels are known. The oxygen and carbon dioxide concentration are measured continuously and accurately as long as an appropriate environment is kept. In case of a deviation in the environment parameters, which are; temperature, humidity and barometric pressure, corrections must be conducted.

ΔN₂ is determined (step 106) by measuring the difference in the nitrogen quantities between the exhaled gas and the inhaled gas during the transition, or by collecting the exhaled gas from the patient during the transition period from the onset of the step change, until the nitrogen concentration level is constant.

Initial concentration levels of N₂ in the lungs (F₀N₂) and final concentration of N₂ in the lungs (F₁N₂) are measured (step 107), ΔN₂ is already known and is used to determine the FRC, by using Equation 3.

$$F R C = \frac{Δ {N}_{2}}{{F}_{0} {N}_{2} - {F}_{1} {N}_{2}} \qquad Equation 3$$

There are several other methods, according to embodiments of the present invention, which may be used to calculate ΔN₂:

The entire volume is collected of exhaled gas in a large reservoir and measuring the volume collected and the nitrogen concentration FN₂ in the bag, by the end of the transition period. According to Equation 4, the volume of the bag times the nitrogen concentration is equal to the total amount of exhaled nitrogen. Subtracting from the amount exhaled nitrogen of the volume of the bag times the inhaled nitrogen concentration, which is the volume of inhaled nitrogen, provides the desired ΔN₂. Rearranging the terms with the bag volume V_{Bag} which is common for both inhalation and exhalation provides Equation 4.

ΔN₂=( F̅_{E}N₂− F̅₁N₂)·V_{Bag} Equation 4

In another method, according to embodiments of the present invention, measuring the exhaled nitrogen concentration F_{E}N₂ continuously. The value of F_{E}N₂ᵢ is measured with each breath following the O₂ concentration step change and is multiplied by the momentary tidal volume VTᵢ. A momentary tidal volume is the momentary exhaled Nitrogen volume. Momentary tidal volume is measured by performing integration on flow. Flow [cc/s or liters per minute] is calculated by using a differential pressure flow transducer or a hot wire anemometer. Subtracting the inhaled nitrogen quantity (FᵢN₂ᵢ·VTᵢ) provides an increment or a decrement of nitrogen in the lung per breath i. The ΔN₂ is then equal to the sum of these increments or decrements over time as given by Equation 5:

$$Δ {N}_{2} = {\sum }_{i = 1}^{∞} ( {F}_{E} {N}_{2 i} - {F}_{I} {N}_{2 i} ) \cdot {VT}_{i} \qquad Equation 5$$

In order to avoid the need to measure nitrogen concentrations and tidal volume for a very long period of time, it is possible to use exponential extrapolation of the nitrogen concentration at the tale end of the step response curve as shown in Equation 6.

$$Δ {N}_{2} = {\sum }_{i = 1}^{n} ( {F}_{E} {N}_{2 i} - {F}_{1} {N}_{2 i} ) \cdot {VT}_{i} + ( {F}_{E} {N}_{2 n} - {F}_{I} {N}_{2 n} ) \cdot {VT}_{n} \cdot {\int }_{t = n \cdot T}^{∞} {e}^{- t τ} d t \qquad Equation 6$$

Where T is the duration of each breath, τ is the time constant of the exponential decay of nitrogen concentration and n is the last breath number where the individual measurements are done.

In another method, according to embodiments of the present invention, calculating of ΔN₂ based on breath-by-breath analysis using a synchronized flow and gas concentration detectors. Thus, the amount of nitrogen in each breath is equal to the momentary product of the exhaled volume V(t) and the nitrogen concentration F_{E}N₂(t) as shown in Equation 7.

ΔN₂=∫(F_{E}N₂(t)−F₁N₂(t))·VT(t)dt Equation 7

Providing approximate calculation by multiplying the alveolar (end-tidal) nitrogen concentration, minus the inspired nitrogen concentration by the tidal volume minus the dead space (which is volume between the upper airways of the patient to the outlet or inlet of a ventilator, as shown in Equation 8:

$$Δ {N}_{2} = {\sum }_{i = 1}^{∞} ( {F}_{ET} {N}_{2 i} - {F}_{I} {N}_{2 i} ) \cdot ( {VT}_{i} - VD ) \qquad Equation 8$$

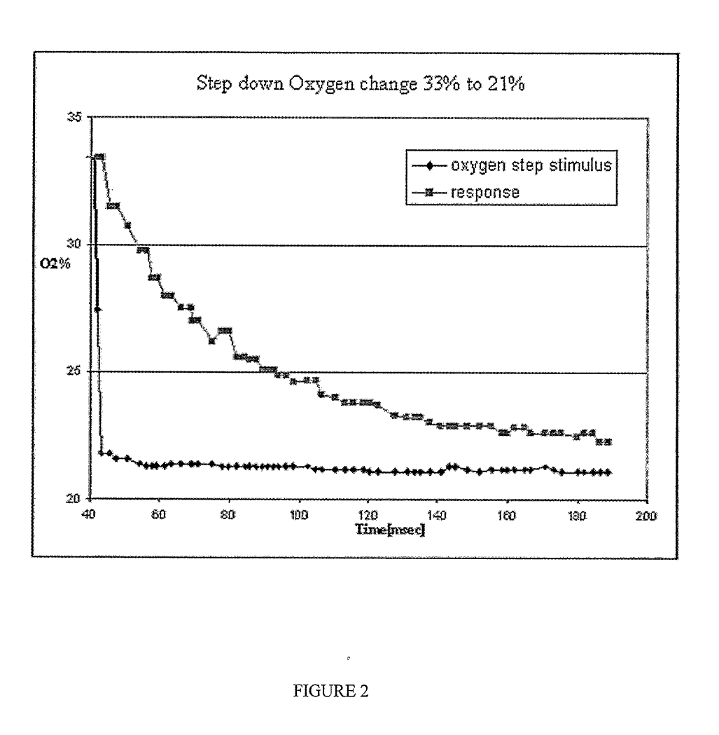

Reference is made to FIG. 2, illustrating, according to an embodiment of the present invention, the step in oxygen in the inhalation (202) and the response to the step of the expiration (201), Oxygen concentration decreases from 35% oxygen to 21% oxygen in the expiration.

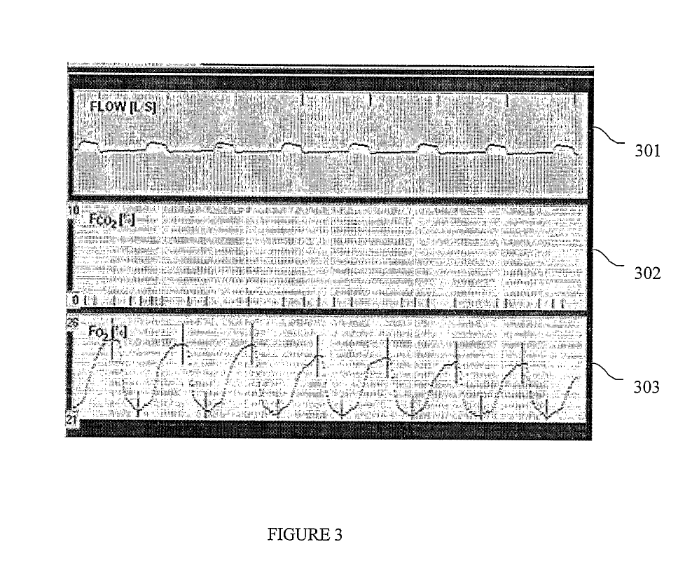

Reference is made to FIG. 3, illustrating, according to an embodiment of the present invention: a monitor reading: a flow reading (301), the volume of air during a predetermined tune, concentration readings (302) of carbon dioxide and concentration readings of oxygen (303). As shown in 302, carbon dioxide readings remains nearly constant.

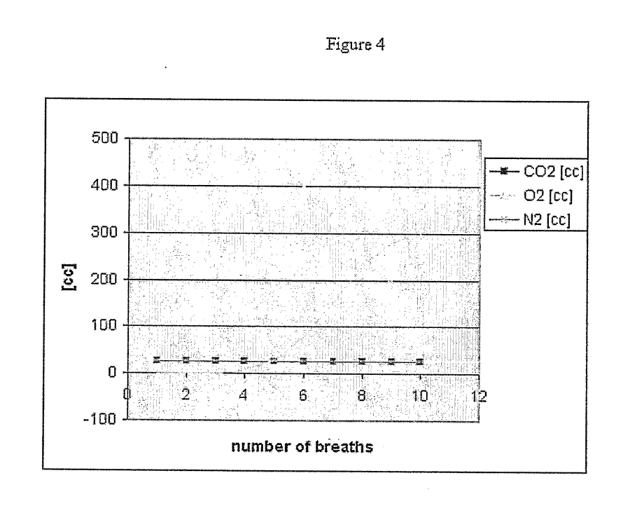

Reference is made to FIG. 4, illustrating a plot of oxygen, carbon dioxide and nitrogen quantity levels, the nitrogen quantity is calculated by using equation 2, according to an embodiment of the present invention. Each dot represents the quantity level of a gas in each breath. The tidal volume value is the summation of the three gas values. The FRC is derived from these values according to the equation 3. Since CO₂ is usually constant, as illustrated in FIG. 4, therefore to a good approximation does not need to be measured continuously in order to calculate FRC.

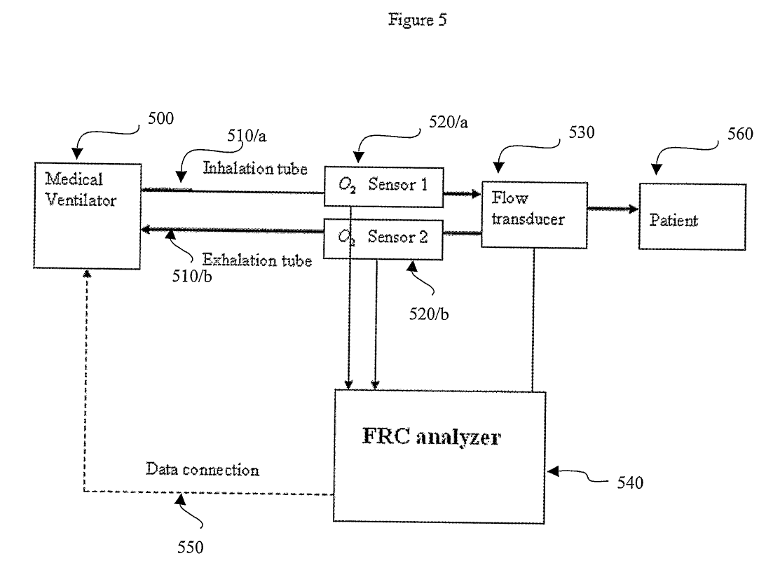

Reference is made to FIG. 5, which illustrates a system for measuring FRC in a schematic diagram, exhibiting three modes of operation, according to embodiments of the present invention. The first mode relates to a manual maneuver for a modifying inspired oxygen, wherein the air flows in an inhalation tube (a tube of inhaled air) (510/a) and an exhalation tube (a tube of exhaled air) (510/b). Oxygen concentration and quantity readings are derived by adding appropriate sensors to each tube. Each sensor (520/a and 520/b) is connected to a respective rube (510/a and 510/b) and measures the oxygen concentration and quantity of the gas flowing through tubes 510/a and 510/b, thereby determining oxygen concentration and quantity of the inhaled and exhaled air. Moreover a transducer (530) for measuring a tidal volume of each breath is connected to both tubes 510/a and 510/b in one end and to the patient's respiratory system (560) from the second end. Both tubes 510/a and 510/b receive air from the patient's respiratory system at one end and to the ventilator 500 from the second end. The ventilator air is a combination mixture of atmospheric air (21% oxygen) and adjusted oxygen concentration (1-100%), providing a mixture with higher oxygen concentration (above 21%), or a lower oxygen concentration (less than 21%). This operation mode is controlled manually, for example by rotating a knob, according to the physician orders.

All readings; the inhaled oxygen concentration, the exhaled oxygen concentration and the tidal volume are collected and analyzed in a special analyzer (540) which calculates the FRC according to the Equations mentioned previously.

The second operation mode, illustrated in FIG. 5 has an additional connection between the FRC device and the ventilator (550). This feature allows tire FRC device to control the oxygen given to the patient, by an integrated control unit which decreases or increases the predetermined parameters. Such parameters may be the levels of O₂%, rate of delivering O₂, duration of O₂ pulses, number of breaths between each pulse of O₂ according to the physician requirements.

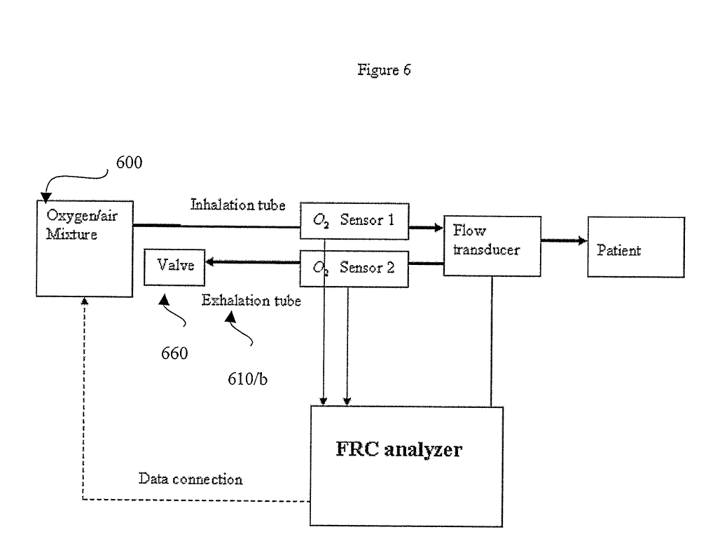

Reference is made to FIG. 6, which illustrates a system for measuring FRC in a schematic diagram similar to FIG. 5, however it relates to a case wherein a patient is able to breath spontaneously, without the ventilator, and provide the step change by a device (600) connected to the patient respiratory system and supply flow of air with the appropriate percentage of oxygen thereby, providing a sudden change in the oxygen percentage to the patient's respiratory system. The device will supply the mixture for inhalation. Exhalation of the patient's breath will be detected by previous discussed sensors and transducer. The exhalation tube (610/b) allows only a flow of air out of the patient's respiratory system and doesn't allow a flow of air to the patient respiratory system, by means such as a valve (660), which allows air flow only in one direction.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

While the invention has been described with respect to a limited number of embodiments, it will be appreciated that many variations, modifications and other applications of the invention may be made.

What is claimed is:

1. In a system for determining functional residual capacity (FRC) of a patient, wherein the patient exhales into the system, thereby providing exhalation to the system and wherein the patient inhales from the system, thereby receiving inhalation from the system, a sensor which measures a fraction of at least one component of the inhalation and a second fraction of the at least one component of the exhalation, the method comprising the steps of:
(a) providing a step change of oxygen fraction of the inhalation;
(b) subsequent to said step change, measuring the fraction of the at least one component in the inhalation and measuring the second fraction of the at least one component in the exhalation; and
(c) calculating the functional residual capacity of the lungs of the patient based on the first fraction and the second fraction.

2. The method, according to claim 1, wherein said providing said step change is performed manually by a technician.

3. The method, according to claim 1, further comprising the step of, and prior to said providing said step change:
(d) programming a programmable device to provide said step change from a medical ventilator to the patient; and
(e) said providing said step change automatically by controlling said medical ventilator with said programmable device.

4. The method, according to claim 1, wherein said step change is an increase of said oxygen fraction.

5. The method, according to claim 1, wherein said step change is a decrease of said oxygen fraction.

6. The method, according to claim 1, wherein an amplitude of the step change is based on the percentage of oxygen provided before the step change.

7. The method, according to claim 1, wherein said step change amplitude is between 25% and 79%.

8. The method, according to claim 1, wherein the duration of said step change is between ten and fifteen breaths of the patient.

9. The method, according to claim 1, further comprising the step of:
(d) providing a second step change wherein the time duration between said step change and said second step change is between three and seven seconds.

10. The method, according to claim 1, wherein said at least one component of the inhalation and said at least one component in the exhalation includes oxygen.

11. The method, according to claim 1, wherein said at least one component of the inhalation and said at least one component in the exhalation includes carbon dioxide.

12. The method, according to claim 1, wherein said at least one component in the inhalation and said at least one component in the exhalation are measured by volumetric flow measurements.

13. The method, according to claim 1, wherein said at least one component includes oxygen, wherein said measuring the fraction of the at least one component in the inhalation and said measuring the second fraction of the at least one component in the exhalation are performed by a sensor for oxygen.

14. The method, according to claim 1, wherein said at least one component includes oxygen, wherein said measuring the fraction of the at least one component in the inhalation and said measuring the second fraction of the at least one component in the exhalation is performed by a sensor for carbon dioxide sensor.

15. The method, according to claim 1, further comprising the step of:
(e) measuring an inhaled volumetric flow and an exhaled volumetric flow by a flow transducer.

16. The method, according to claim 1, further comprising the step of:
(f) measuring said an inhaled volumetric flow and an exhaled volumetric flow by a hot wire anemometer.

17. The method, according to claim 1, wherein said calculating said functional residual capacity of the lungs includes calculating initial and final nitrogen volume fractions.

18. The method, according to claim 1, wherein said calculating said functional residual capacity of the lungs is perforated by calculating a tidal volume of a breath of the patient.

19. The method, according to claim 18, wherein said calculating said tidal volume is derived from said volumetric flow measurements.

20. The method, according to claim 19, wherein initial and final nitrogen concentrations FN₂ are calculated according to equation: FN₂=0.99−(FO₂+FCO₂),
wherein FCO₂ is a carbon dioxide concentration, FO₂ is an oxygen concentration, FN₂ is a nitrogen concentration.

21. The method, according to claim 20, wherein said initial and final nitrogen concentrations are determined by measuring an end tidal nitrogen concentration.

22. The method, according to claim 1, wherein calculating said functional residual capacity of the lungs includes calculating initial and final nitrogen quantities.

23. The method, according to claim 1, wherein calculating the functional residual capacity includes calculating the difference between an exhaled nitrogen quantity and an inhaled nitrogen quantity.

24. The method, according to claim 23, wherein calculating the difference between the exhaled nitrogen quantity and the inhaled nitrogen quantities includes measuring a nitrogen quantity difference between the exhalation and inhalation during a transition.

25. The method, according to claim 23, wherein calculating the difference between the exhaled nitrogen quantity and the inhaled nitrogen quantities includes collecting exhaled nitrogen, during a transition, from the onset of said step until the nitrogen level is constant.

26. A system for determining functional residual capacity (FRC) of a patient, the system comprising:
(a) a medical device which provides inhalation to the patient.
(b) a sensor which measures a fraction of at least one component of the inhalation and a second fraction of said at least one component of the exhalation;
wherein a step change of oxygen fraction of the inhalation is provided to the patient and subsequent to said step change, the fraction of the at least one component in the inhalation is measured and the second fraction of the at least one component in the exhalation is measured; and
wherein the functional residual capacity of the lungs of the patient is calculated based on the first fraction and the second fraction.

27. The system, according to claim 26, further comprising:
(c) a programmable device which provides said step change to a medical ventilator.

28. The system, according to claim 26, wherein said programmable device receives a signal as output of said sensor, and based on said output and said step change calculates said functional residual capacity.

29. The programmable device, according to claim 27.

30. The system, according to claim 26 wherein the medical device is integrated with a medical ventilator.
